(19) United States
(12) Patent Application Publication
(10) Pub. No.: US 2014/0114130 A1
(43) Pub. Date: Apr. 24, 2014

(54) POWER SUPPLY APPARATUS FOR LIGHT SOURCE OF ENDOSCOPE AND ENDOSCOPIC SYSTEM HAVING POWER SUPPLY APPARATUS FOR LIGHT SOURCE OF ENDOSCOPE

(71) Applicant: OLYMPUS CORPORATION, Tokyo (JP)

(72) Inventor: Toshiro IJICHI, Hachioji-shi (JP)

(73) Assignee: OLYMPUS CORPORATION, Tokyo (JP)

(21) Appl. No.: 14/078,968

(22) Filed: Nov. 13, 2013

Related U.S. Application Data
(63) Continuation of application No. PCT/JP2013/063652, filed on May 16, 2013.

(30) Foreign Application Priority Data
May 16, 2012 (JP) 2012-112280

(51) Int. Cl. H05B 37/02; A61B 1/06

(52) U.S. Cl. CPC H05B 37/02; A61B 1/0661
USPC 600/180; 315/291

(57) ABSTRACT

A power supply apparatus for a light source of an endoscope includes a light source unit including an input portion that a control signal is input from an output control unit, the light source unit switching a brightness of the illumination light by the control signal and a control signal cable connected to the output control unit and the input portion, and including a current control signal line to transmit the control signal, and a GND line. The power supply apparatus for a light source of an endoscope further includes a common-mode rejecting portion provided in the input portion, connected to the control signal cable, and rejects noise superimposed on the control signal cable.

### CROSS-REFERENCE TO RELATED APPLICATIONS

This application is a Continuation Application of PCT Application No. PCT/JP2013/063652, filed May 16, 2013 and based upon and claiming the benefit of priority from prior Japanese Patent Application No. 2012-112280, filed May 16, 2012, the entire contents of all of which are incorporated herein by reference.

### BACKGROUND OF THE INVENTION

1. Field of the Invention The present invention relates to a power supply apparatus for a light source of an endoscope, which is arranged for a light source apparatus of an endoscope, and an endoscopic system having the power supply apparatus for a light source of an endoscope.

2. Description of the Related Art

A power supply apparatus for light source of endoscope have a function of switching an output current to the light source apparatus, to change a brightness of illumination light. So, for example, a relay circuit or a switching circuit is used, and a set value is selected. Thereby, the output current is switched. Specifically, the light source apparatus switches the output current to one of preset fixed values, such as 18A, 19A, and 20A. In the switching operation, a control signal for switching instruction is a digital control signal to drive the relay circuit and the switching circuit. A technique of changing the brightness of the illumination light with such a digital signal is disclosed in, for example, Jpn. Pat. Appln. KOKAI Pub. No. 2005-237430.

### BRIEF SUMMARY OF THE INVENTION

An aspect of a power supply apparatus for a light source according to the present invention comprises: a light source unit including a control signal input portion that an analogue control signal is input from an output control unit that outputs the control signal to control a light source portion that emits illumination light, the light source unit switching a brightness of the illumination light by the control signal; a control signal cable connected to the output control unit and the control signal input portion, and including a current control signal line to transmit the control signal, and a GND line; and a common-mode rejecting portion provided in the control signal input portion, and rejecting noise superimposed on the control signal cable.

An aspect of an endoscopic system comprises: a light source portion emitting illumination light; an output control unit outputting an analogue control signal to control the light source portion; and the power supply apparatus for a light source of an endoscope described above.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEW OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic structural diagram of an endoscopic system according to a present invention.

FIG. 2 is a diagram illustrating a circuit configuration of a power supply apparatus according to a first embodiment.

FIG. 3 is a diagram illustrating a circuit configuration of a power supply apparatus according to a first modification of the first embodiment.

FIG. 4 is a diagram illustrating a circuit configuration of a power supply apparatus according to a second modification of the first embodiment.

### DETAILED DESCRIPTION OF THE INVENTION

Embodiments of the present invention will be explained in detail hereinafter with reference to the drawings.

### First Embodiment

[Structure]

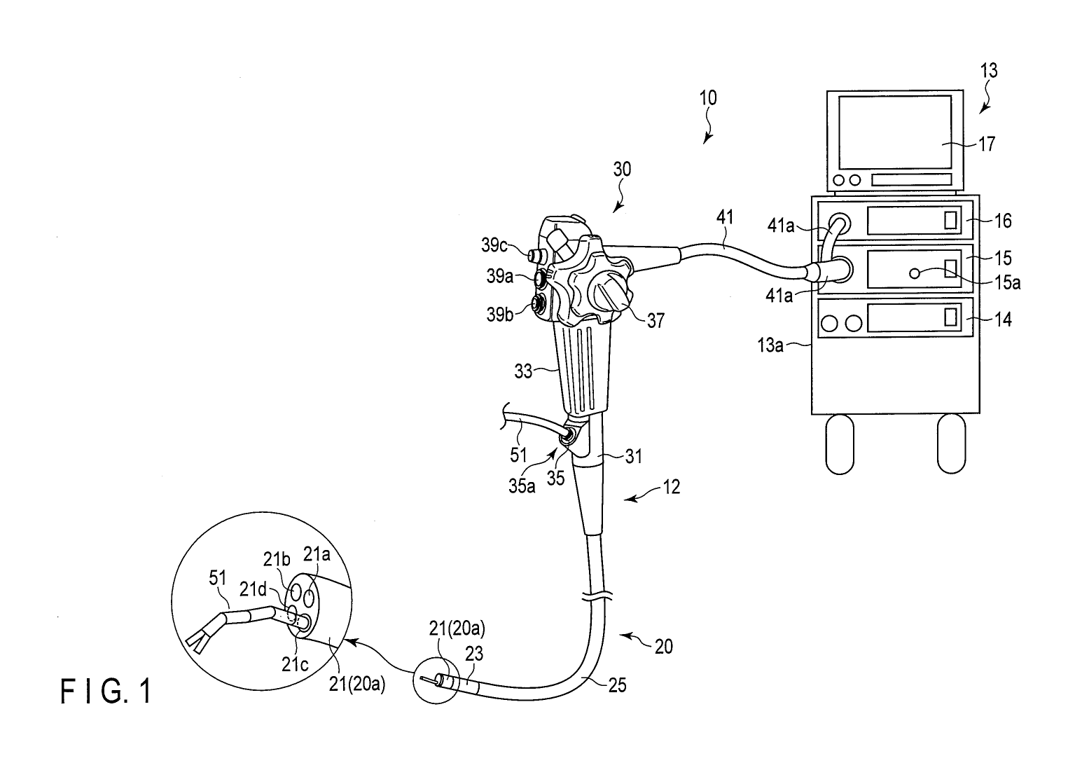

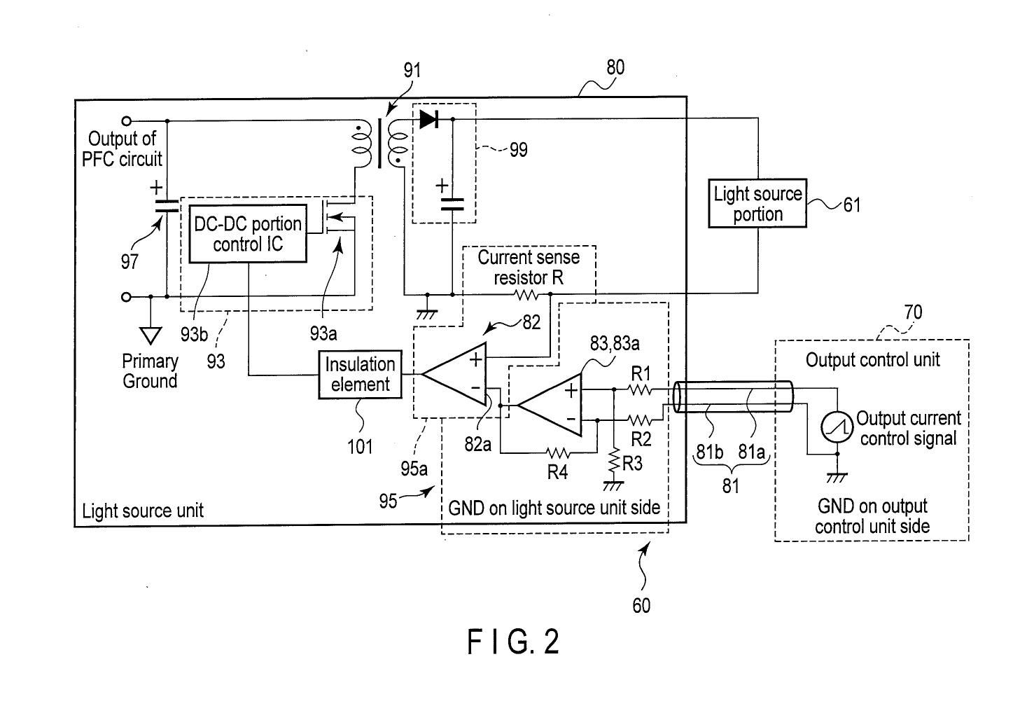

A first embodiment will be explained hereinafter with reference to FIG. 1 and FIG. 2.

[Endoscopic System 10]

As illustrated in FIG. 1, an endoscopic system 10 comprises an endoscope 12 that images a desired object to be observed, and peripheral equipment 13 of the endoscope 12.

[Peripheral Equipment 13]

The peripheral equipment 13 is mounted onto a hand cart 13. The peripheral equipment 13 includes a control apparatus 14 (for example, a video processor) that performs control and arithmetic processing for the whole endoscopic system 10, and a light source apparatus 15 that generates illumination light. The peripheral equipment 13 also includes an image process apparatus 16 that performs various types of image processing for images imaged by an imaging portion (not shown) of the endoscope 12, and an image display apparatus 17 that displays images, image data (images that have been subjected to image processing performed by an process apparatus 4), the state of the apparatus, and the operation instruction state.

The control apparatus 14 is connected to the light source apparatus 15, the image process apparatus 16, and the image display apparatus 17, and controls them.

The light source apparatus 15 and the image process apparatus 16 are detachably connected to a connector 41a (described below) of the endoscope 12. The light source apparatus 15 and the image process apparatus 16 are connected to the image display apparatus 17.

The light source apparatus 15 includes an operating portion 15a, such as a knob, to operate a light source portion 61 described below. The operating amount of the operating portion 15a is switched a brightness of the light source portion. In other words, the operating portion 15a adjusts the light intensity of the illumination light that the light source portion emits.

The image display apparatus 17 displays, for example, an object to be observed which has been imaged by the imaging portion. The image display apparatus 17 includes, for example, a monitor. The object to be observed is, for example, the affected part or the lesion part in the test subject (for example, the body cavity).

[Endoscope 12]

As illustrated in FIG. 1, the endoscope 12 includes a hollow and elongated insertion portion 20 to be inserted into the body cavity of the patient, and an operating portion 30 that is connected to a proximal end portion of the insertion portion 20 and operates the endoscope 12.

[Insertion Portion 20]

As illustrated in FIG. 1, the insertion portion 20 includes a distal end hard portion 21, a bending portion 23, and a flexible tube portion 25, arranged in this order from the side of the distal end portion 20a of the insertion portion 20 toward the proximal end portion of the insertion portion 20. A proximal end portion of the distal end hard portion 21 is coupled to a distal end portion of the bending portion 23, and a proximal end portion of the bending portion 23 is coupled to a proximal end portion of the flexible tube portion 25.

The distal end hard portion 21 functions as the distal end portion 20a of the insertion portion 20 and a distal end portion of the endoscope 12, is hard and cannot be bent. The distal end hard portion 21 includes the imaging portion (not shown) that images, for example, the object to be observed. The imaging portion includes, for example, a CMOS image element. The distal end hard portion 21 also includes an observation window 21a provided in front of the imaging portion, an illumination window 21b through which the illumination light is emitted, and an opening portion 21c, from which the endoscopic treatment tool 51 projects, and communicating with the treatment-tool insertion channel 35.

The bending portion 23 is bent in a desired direction, such as up and down, and left and right, by operation of a bending operation portion 37 described below. When the bending portion 23 is bent, the position and the direction of the distal end hard portion 21 are change, thus the object to be observed comes within the observed range of the endoscope, and the illumination light is applied to the object to be observed.

The flexible tube portion 25 has a desired flexibility. The flexible tube portion 25 is bent by external force. The flexible tube portion 25 is a tube-like member extending from a main body portion 31 (described below) of the operating portion 30.

[Operating Portion 30]

As illustrated in FIG. 1, the operating portion 30 includes a main body portion 31, from which the flexible tube portion 25 extends, a grasping portion 33 to be coupled to the proximal end portion of the main body portion 31 and to be grasped by the operator who operates the endoscope 12, and a universal cord 41 connected to the grasping portion 33.

[Main Body Portion 31]

The main body portion 31 includes a treatment-tool insertion port 35a. The treatment-tool insertion port 35a is coupled to the proximal end portion of the treatment-tool insertion channel 35. The treatment-tool insertion channel 35 is provided to extend from the flexible tube portion 25 to the distal end hard portion 21 in the insertion portion 20. A distal end portion of the treatment-tool insertion channel 35 communicates with the opening portion 21c provided in the distal end hard portion 21. The treatment-tool insertion port 35a is an insertion port for inserting the endoscopic treatment tool 51 into the treatment-tool insertion channel 35. The endoscopic treatment tool 51 is inserted from the treatment-tool insertion port 35a into the treatment-tool insertion channel 35, and pushed to the side of the distal end hard portion 21. Then, the endoscopic treatment tool 51 projects from the opening portion 21c. The endoscopic treatment tool 51 includes, for example, an ultrasonic scalpel, or a high-frequency knife.

[Grasping Portion 33]

The grasping portion 33 includes the bending operation portion 37 that bends and operates the bending portion 23.

The grasping portion 33 also includes a switch part 39 operated by the operator's hand when the main body portion 31 is grasped by the operator. The switch part 39 includes an suction switch 39a, and an air/water feed switch 39b. The suction switch 39a is operated when the endoscope 12 sucks mucus or fluid from the opening portion 21c also serving as an suction opening part provided in the distal end hard portion 21 through the treatment-tool insertion channel also serving as an suction channel. The air/water feed switch 39b is operated when air/liquid fluid is fed through an air/water feed channel (not shown), to secure the observation field (observation window 21b) of the imaging portion in the distal end hard portion 21. The air/water feed channel is inserted through the universal cord 41, the operating portion 30, and the insertion portion 20. The distal end portion of the air/water feed channel communicates with the opening portion 21d opened in the distal end hard portion 21. The fluid includes water and gas.

The switch part 39 includes various operating part 39c.

[Universal Cord 41]

The universal cord 41 extends from a side surface of the grasping portion 33. An end portion of the universal cord 41 is branched into two, and connectors 41a are arranged for the respective branches. One of the connectors 41a can be attached to and detached from the light source apparatus 15, and the other of the connectors 41a can be attached to and detached from the image process apparatus 16.

[Power Supply Apparatus 60 for Light Source]

As illustrated in FIG. 2, the endoscopic system 10 includes a power supply apparatus 60 for light source of the endoscope 12 which is provided in the light source apparatus 15, switches the brightness of illumination light on the basis of the output current that linearly changes, and thereby controls the brightness of the illumination light. The output current that linearly changes includes, for example, an analogue control signal.

The power supply apparatus 60 for light source includes a light source portion 61 that emits illumination light, and an output control unit 70 that outputs an analogue control signal included in the output current to control the light source portion 61. The power supply apparatus 60 for light source also includes a light source unit 80 that includes the light source 61, controls the light source portion 61 by an analogue control signal output from an output control unit 70, switches the illumination light by the analogue control signal, and thereby controls the brightness of the illumination light.

The power supply apparatus 60 for light source also includes a control signal cable 81 connected to the output control unit 70 and an input portion 82a (described below) of the light source unit 80. The control signal cable 81 includes a current control signal line 81a to transmit an analogue control signal from the output control unit 70 to the input portion 82a, and a GND line 81b.

[Control Signal Cable 81]

The control signal cable 81 is covered with a shield (not shown) to prevent the control signal cable 81 from being influenced by noise generated from the endoscopic treatment tool 51 being an external device.

The output control unit 70 and the light source unit 80 are electrically connected with each other by the control signal cable 81 described below. The output control unit 70 and the light source unit 80 are mounted onto the same base member, such as a chassis, such that their GNDs have the same potential level, and are connected to a common GND.

[Light Source Portion 61]

The light source portion 61 includes, for example, a discharge lamp. The light source portion 61 is connected to a light guide (not shown). The light guide is provided inside the universal cord 41, the grasping portion 33, the main body portion 31, and the insertion portion 20, and connected to the illumination window 21b. Illumination light emitted from the light source portion 61 is guided by the light guide, and emitted from the illumination window 21b.

[Output Control Unit 70]

The output control unit 70 is connected to the operating portion 15a. The output amount of the output current, more specifically, the value of the analogue control signal varies according to the operation amount of the operating portion 15a. The output control unit 70 outputs an output current with an output amount varied in accordance with the operation amount of the operating portion 15a. The output control unit 70 also controls the light source portion 61 by the output current.

[Structure 1 of Light Source Unit 80]

The light source unit 80 includes an error amplifier 82 including an input portion 82a which is a control signal input portion that an analogue control signal is input from the output control unit 70.

The light source unit 80 also includes a common-mode rejecting portion 83 that is provided in the input portion 82a that an analogue control signal is input, connected to the current control signal line 81a and the GND line 81b, and rejects noise superimposed on the current control signal line 81a and the GND line 81b.

[Common-mode Rejecting Portion 83, Differential Amplifier Circuit 83a]

The common-mode rejecting portion 83 includes, for example, a differential amplifier circuit 83a. When the control signal cable 81 receives noise generated from, for example, the endoscopic treatment tool 51 which is an external device, the noise is superimposed on the current control signal line 81a and the GND line 81b. In the current control signal line 81a and the GND line 81b, the noise has the common mode and the same level, and is a common-mode noise. The differential amplifier circuit 83a rejects the noise when an analogue control signal, on which the noise is superimposed, is input to the input portion 82a.

In the differential amplifier circuit 83a, the noise rejection property depends on the common mode rejection ratio (hereinafter referred to as CMRR) of the operation amplifier used. The CMRR is generally, for example, about 80 dB.

It is assumed that resistors of the differential amplifier 83a are R1, R2, R3, and R4, the resistance of R1 is equal to the resistance of R2, and the resistance of R3 is equal to the resistance of R4. R1, R2, R3, and R4 are a resistor array.

An input end of the differential amplifier 83a of the common-mode rejecting portion 83 is electrically connected to the control signal cable 81 via the resistors R1 and R2. Thereby, the analogue control signal output from the output control unit 70 is input to the differential amplifier 83a. The differential amplifier circuit 83a is grounded via the resistor R3 that is electrically connected to the input end (+) of the differential amplifier circuit 83a.

[Structure 2 of Light Source Unit 80]

The light source unit 80 also includes a transformer unit 91, a control portion 93 provided on a primary side of the transformer unit 91, a control signal input portion 95, a smoothing element 97, and a rectification circuit 99 provided on a secondary side of the transformer unit 91.

[Transformer Unit 91]

The transformer unit 91 is a unit forming an amplifying portion. The amplifying portion mainly includes, for example, a transformer with a transformation ratio (current ratio) according to the design.

[Control Portion 93]

The control portion 93 includes a current control element 93a connected to a primary-side line of the transformer unit 91, and a DC-DC portion control IC 93b being a control apparatus that controls the current control element 93a. The current control element 93a includes, for example, an NchFET.

[Control Signal Input Portion 95]

The control signal input portion 95 is electrically connected to the output control unit 70 via the control signal cable 81. An analogue control signal is input through the control signal cable 81 to the control signal input portion 95. The control signal input portion 95 includes an error detecting portion 95a formed of the error amplifier 82 and a current sense resistor R, and the common-mode rejecting portion 83.

In the error detecting portion 95a, a secondary-side current generated by the current sense resistor R and the output current of the differential amplifier circuit 83a is input an input end of the error amplifier 82. The current sense resistor R is connected to a secondary-side line of the transformer unit 91.

The control signal input portion 95 is connected to an input side of the control portion 93 via an insulation element 101 to protect the circuit. A difference is output from an output end of the error amplifier 82 to the control portion 93 through the insulation element 101. The insulation element 101 is an element that electrically insulates the input from the output by performing signal transmission with light by using a photo coupler element or the like, to isolate the circuit that may be touched by the operator of the transformer unit 91 or the patient from the primary circuit connected to a commercial power supply.

[Function]

When the operating portion 15a of the light source apparatus 15 is operated, the output control unit 70 outputs an analogue control signal which is an output current, in accordance with the operating amount. The analogue control signal flows through the current control signal line 81a of the control signal cable 81.

In the operation, part of the noise generated by the endoscopic treatment tool 51 being an external device or the like is superimposed on the current control signal line 81a and the GND line 81b. The other part of the noise is removed by a shield (not shown).

The noise superimposed on the analogue control signal is rejected from the analogue control signal by the differential amplifier circuit 83a when the analogue control signal is input to the input portion 82a.

In addition, since the resistors R1, R2, R3, and R4 are a resistor array, variations in resistance are suppressed, and more noise is rejected.

The light source portion 61 emits illumination light in response to the analogue control signal flowing from the error amplifier 82. The brightness of the illumination light is smoothly switched by the analogue control signal that varies in accordance with the operation amount of the operating portion 15a. Thereby, the illumination light is switched in accordance with and in response to the operation of the operating portion 15a.

[Effect]

As described above, according to the present embodiment, the light source portion 61 is controlled by the analogue control signal, and thereby the brightness of the illumination light is smoothly switched. Thereby, the present embodiment enables improvement in the following capability and reactivity in comparison with the case of using a digital signal when the brightness of the illumination light is switched, and switching of the brightness of the illumination light without obstruction to the switching operation for the illumination light. Thus, the present embodiment enables setting of the proper brightness even when the light transmittance of the endoscope 12 differs according to the type of the endoscope, and provides optimum brightness according to the brightness desired by the operator and the conditions related to the object to be observed.

In addition, according to the present embodiment, tolerance to noise is securely improved, since noise is rejected from the analogue control signal by the differential amplifier 83a.

Further, according to the present embodiment, the brightness of the illumination light is smoothly switched by the differential amplifier circuit 83a, without influence of an external device.

In addition, the present embodiment prevents the analogue control signal in a state where the rejected noise is superimposed on the analogue control signal again from being input to the input portion 82a, by virtue of the differential amplifier circuit 83a provided in the input portion 82a.

Further, according to the present embodiment, a resistor array is used as resistors of the differential amplifier circuit 83a, and thus variations in resistance is suppressed, and more noise can be rejected.

[First Modification]

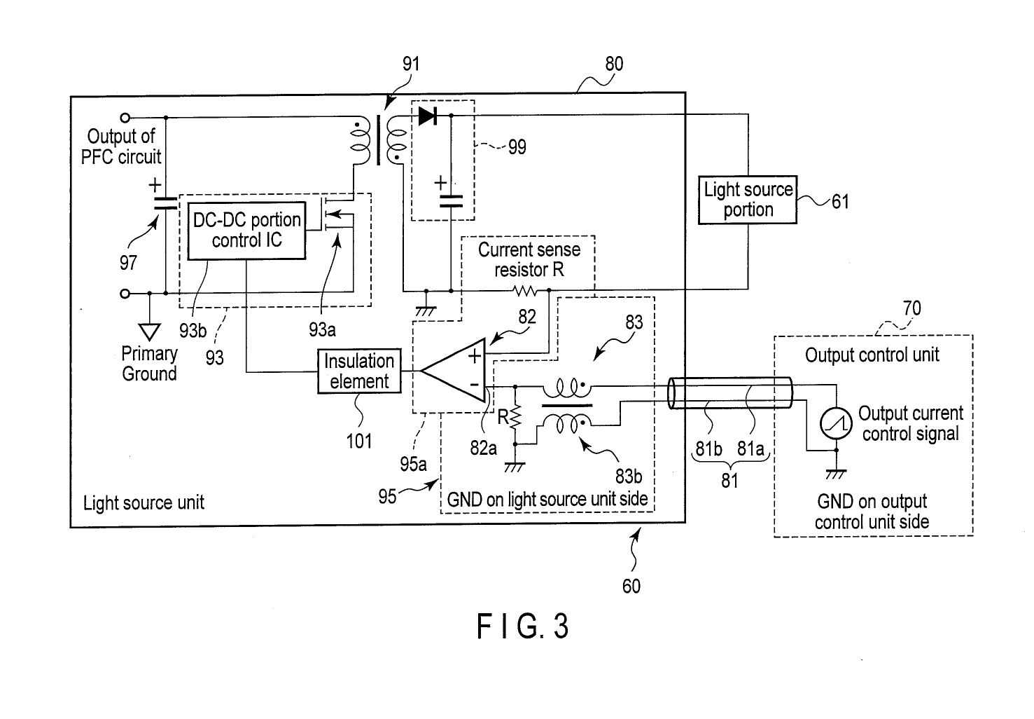

As illustrated in FIG. 3, the common-mode rejecting portion 83 may include a common-mode choke coil 83b. In this case, the power supply apparatus 60 for light source can be manufactured at lower cost than that in the first embodiment.

[Second Modification]

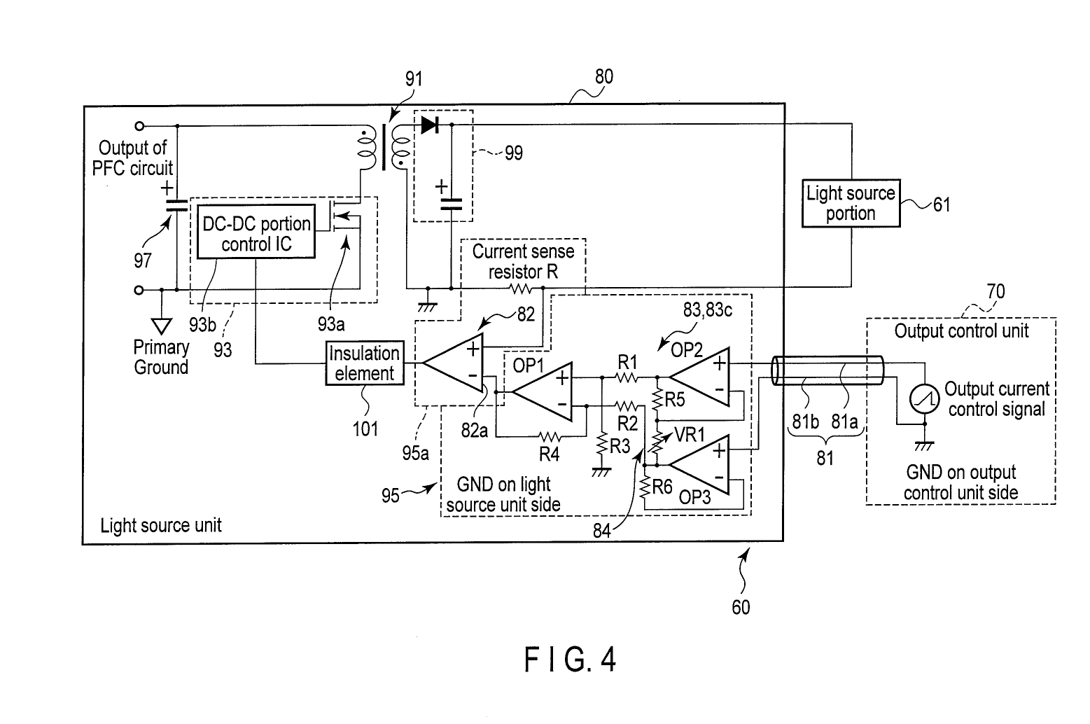

As illustrated in FIG. 4, the common-mode rejecting portion 83 may include an instrumentation circuit 83c. In this case, the modification achieves a CMRR improved more than that in the first embodiment, and more noise can be rejected.

The power supply apparatus 60 for light source may include a variable resistor 84 to adjust the balance between the OP2 and the OP3.

The present invention is not limited to the above embodiment, and may be carried out by modifying the constituent elements within a range not departing from the gist. Various inventions can be made by proper combinations of constituent element disclosed in the above embodiment.

Additional advantages and modifications will readily occur to those skilled in the art. Therefore, the invention in its broader aspects is not limited to the specific details and representative embodiments shown and described herein. Accordingly, various modifications may be made without departing from the spirit or scope and the general inventive concept as defined by the appended claims and their equivalents.

What is claimed is:

1. A power supply apparatus for a light source of an endoscope, comprising:
a light source unit including a control signal input portion that an analogue control signal is input from an output control unit that outputs the control signal to control a light source portion that emits illumination light, the light source unit switching a brightness of the illumination light by the control signal;
a control signal cable connected to the output control unit and the control signal input portion, and including a current control signal line to transmit the control signal, and a GND line; and
a common-mode rejecting portion provided in the control signal input portion, and rejects noise superimposed on the control signal cable.

2. The power supply apparatus for a light source of an endoscope, according to claim 1, wherein
the common-mode rejecting portion includes a differential amplifier circuit.

3. The power supply apparatus for a light source of an endoscope, according to claim 1, wherein
the common-mode rejecting portion includes a common-mode choke coil.

4. The power supply apparatus for a light source of an endoscope, according to claim 1, wherein
the common-mode rejecting portion includes an instrumentation circuit.

5. An endoscopic system comprising:
a light source portion emitting illumination light;
an output control unit outputting an analogue control signal to control the light source portion; and
the power supply apparatus for a light source of an endoscope according to claim 1.
